(19) United States
(12) Patent Application Publication
(10) Pub. No.: US 2005/0003556 A1
(43) Pub. Date: Jan. 6, 2005

(54) Probe Beads for affirnity reaction and detection system

(76) Inventors: Hiroshi Nagasawa, Kanagawa (JP); Masayoshi Hirose, Kanagawa (JP); Akira Fukunaga, Kanagawa (JP)

Correspondence Address: WENDEROTH, LIND & PONACK, L.L.P., 2033 K STREET N. W., SUITE 800, WASHINGTON, DC, 20006-1021, US

(21) Appl. No.: 10/493,017

(22) PCT Filed: Oct. 31, 2002
(86) PCT No.: PCT/JP02/11386

(30) Foreign Application Priority Data
Oct. 31, 2001 (JP) 2001-334341

(51) Int. Cl. G01N 33/543

(52) U.S. Cl. 436/518; 435/6

(57) ABSTRACT

The present invention discloses affinity reaction probe beads having a structure comprising probe molecules immobilized on porous bead carriers provided with one or more individual identification signals among individual identification signals including optical signals such as digital signals using optical graphics, for example, bar codes or dot matrix bar codes, and color signals based on color information; and radio or electronic signals which issue individual information, such as IC tags, or tuning circuits or oscillation circuits for radio waves or electricity, a method for producing the affinity reaction probe beads, and an analyte detection system using the affinity reaction probe beads. By using the affinity reaction probe beads, the invention provides a reaction detection system which can be utilized for various physiological function diagnoses such as a diagnosis of single-nucleotide polymorphism (SNPs).

### TECHNICAL FIELD

This invention relates to affinity reaction probe beads which enable the recognition of many functional molecules for use, for example, in genetic diagnosis and physiological function diagnosis; a method for producing them; and a detection system using them.

### BACKGROUND ART

Affinity detection, which uses a substance selectively binding to a particular molecule to detect a corresponding substance selectively, is a very sensitive method of detection. This method has been used, for example, in a liquid chromatograph as an affinity column for detecting a particular protein with the use of a particular enzyme. However, this affinity detection method in liquid chromatography merely gives information on a single particular molecule or a small number of particular molecules, and is not an analytical means for giving information on the presence of many molecules at the same time.

Detection of polymorphism due to mutation of a gene, especially mutation of one base (sequence), is effective for diagnosis of a disease attributed to mutation or the like, for example, diagnosis of cancer. This detection is also necessary for guidelines on drug response and side effects, and contributes to the analysis of genes related to causes of multifactorial diseases as well as to predictive medical care.

Currently, the use of a so-called DNA microarray, one of affinity methods, is known to be effective for this detection. The DNA microarrays are roughly divided into DNA microarrays in a narrow sense in which natural or synthetic oligonucleotides, or cDNA's obtained by reverse transcription from mRNA's have been immobilized, as probes, on a substrate of glass or the like; and DNA chips in which oligonucleotides have been synthesized on a substrate by photolithography technology. These DNA microarrays are used as analytical means by having test samples brought into contact with them to hybridize objects of analysis with the probes, thereby obtaining information on the existence of many molecules, as objects of analysis, at the same time.

Affymetrix's Gene Chip utilized so far, a DNA chip having short synthetic DNA strands, usually has more than 10,000 different oligoDNA fragments (DNA probes) synthesized within 1 cm square on a silicon or glass substrate by photolithography technology. When a DNA sample (e.g. fluorescence-labeled one) to be examined is flowed on this DNA chip, DNA fragments having sequences complementary to the probes on the DNA chip bind to these probes, and only the bound portions can be distinguished by fluorescence. Thus, particular sequences of the DNA fragments in the DNA sample can be recognized and determined. This method has already been shown to be capable of detecting mutations in oncogenes, or detecting genetic polymorphism (for example, single-nucleotide polymorphism).

A DNA microarray, having cDNA's arrayed on a slide glass, is also used. In preparing the DNA microarray, the first step is to have several thousand to several million cDNA's ready for use. Clones of cDNA's are acquired, and the cDNA's are prepared by PCR. To secure such numerous cDNA's, a lot of time and huge expenses are required.

These affinity reaction probe methods involving chips are very effective as analytical means giving information on the existence of many molecules at the same time, but posed several problems. For example, the DNA chip using photolithography needs at least 4 photomasks for one stage of synthesis (addition of one nucleotide) in synthesizing an oligonucleotide. Moreover, a procedure comprising 4 cycles of photolithography, coupling and washing is repeated to cover the required chain length, thereby resulting in a high cost. To change the pattern of the synthetic oligonucleotide, it is necessary to replace the photomasks. Thus, it was impossible to prepare DNA chips of various designs flexibly fulfilling needs. In addition, synthesis took place while using one photomask at a time and performing one stage at a time, so that the grade of each spot was not ensured, thus posing the problem that chip-to-chip reproducibility remained insecure.

A DNA microarray prepared by spotting pre-synthesized oligonucleotide solutions at high densities has been proposed as an alternative method. This DNA microarray makes changing of the pattern easier than the method using photolithography. However, the step of spotting probe molecules, spot by spot, onto immobilization glass or the like must be carried out, thus presenting the drawback of leading to a high cost as does the DNA chip using photolithography.

At the time of detection by reaction chips comprising these DNA microarrays, moreover, hybridization was localized, and definitive determination was not possible any more. Thus, it was necessary to use a dedicated device for hybridization, and perform a long-term reaction. Hence, detection of various pieces of genetic information, including those on bone marrow transplantation, involved enormous labor, took much time, and cost heavy expenses.

In recent years, proposals have been made of techniques which detect analytes with the use of DNA resin beads having cDNA's immobilized on resin beads using particular colors as identification marks. According to these techniques, the carrier beads are individually identified by combinations of the types and densities of colors. However, in order to give identifying information necessary for analysis of an object to be analyzed, for example, single-nucleotide polymorphism (SNP), several tens of thousands of identifications need to be made, but only the identifications in the several hundred range can be made. Furthermore, the identifications based on analog information, such as information on the types and densities of colors, cannot completely avoid read errors.

Besides, the proposed carrier resin beads are solid-core materials and, thus, probes are immobilized on the surface of the beads. The probes so bound to the surface of beads are susceptible to external physical and chemical influences, and the probes are prone to fall off or deteriorate, making them unstable. Since immobilization has taken place only on the surface of the beads, moreover, there is the drawback that the probe binding area of the carrier is small.

### DISCLOSURE OF THE INVENTION

The present invention aims at establishing a more convenient detection system and providing a reaction detection system which can be used for various physiological function diagnoses such as a diagnosis of single-nucleotide polymorphism (SNP), and an affinity reaction probe for use in the reaction detection system.

We, the present inventors, considered the aforementioned problems, and conducted various studies of materials and forms of reaction probe chips which do not use photolithography technology—a technology involving a lengthy and complicated reaction process, having difficulty in flexibly attaining different purposes, and entailing a huge cost—and do not use a dedicated device for hybridization, but nevertheless have on their surface a high integration degree comparable to that obtained with the use of photolithography equipment.

These studies led to the finding that a reaction probe chip can be constructed in a virtual space, if advanced individual detection information is available in the detection of hybridization. As an individual identification signal, it is preferred to use, aside from fluorescence for use on the object of detection, an identification signal capable of carrying a large amount of information, such as optical information, for example, graphic information such as a bar code or a dot matrix, or color information, or radio or electronic information from an IC tag or a radio wave or electricity tuning circuit or oscillation circuit. Of course, the use of information on the specific gravity, particle diameter, presence or absence of magnetism, of beads, or any other information as an identification signal is not precluded. We have paid attention to the fact that if respective carrier beads can be individually identified, the aforementioned problems can be resolved by a simple method which comprises putting an object to be detected, into a reactor charged randomly with beads on which probe molecules having a detecting capacity have been immobilized. Based on this fact, we have arrived at the present invention.

According to the present invention, porous beads are used as carriers, whereby the surface area usable for immobilization of probes is large relative to the size of the beads, so that large amounts of probes can be bound to the beads. Moreover, the probes are immobilized on the internal surfaces of the pores within the carrier. This confers the advantage that the probes are unsusceptible to physical and chemical influences, and are stable to deterioration or falling-off.

### BRIEF DESCRIPTION OF THE DRAWING

FIGS. 1(a) and 1(b) are overall image views of an affinity reaction probe bead having a reactive probe molecule immobilized therein, FIG. 1(a) showing a bar code of a dot matrix printed in a part of a porous glass bead, and FIG. 1(b) showing an electronic identification circuit printed on a single crystal sphere of silicon.

FIG. 2 is a view showing the concept of a method for writing a dot matrix mark.

FIG. 3 is a view showing the concept of a method for producing a carrier.

FIG. 4 is a conceptual view showing the immobilization of the probe molecules, cDNA's, onto the inner surface of the carrier.

FIG. 5 is a conceptual view showing the surface synthesis of the probe molecules, oligonucleotide DNA's, onto the inner surface of the carrier.

FIG. 6 is an explanatory view of the flow of steps in a detection system, including a hybridization device and a fluorescence observer, as an affinity reaction probe bead detection system.

The numerals or symbols in the drawings represent the following:
1 Carrier
1A Porous carrier
1B Single crystal sphere-shaped silicon carrier
2 Dot matrix
3 Electronic identification circuit (ROM circuit)
4 Silica membrane (SiO₂ coating)
5 Probe molecule
6 Linker
7 Base block
8 Affinity reaction probe bead
8A Reaction probe bead reacted
9 Reaction tube
10 Fluorescence-labeled sample (fluorescence-labeled cDNA) (sample)
11 Fluorescence observer
12 Identification mark discriminator
13 Data processor
   [Embodiments of the Invention]

The present invention has solved the aforementioned problems by the following means:

(1) Affinity reaction probe beads having a structure comprising probe molecules immobilized in porous carrier beads provided with one or more individual identification signals among individual identification signals including optical signals such as digital signals using optical graphics, for example, bar codes or dot matrix bar codes, and color signals utilizing the types and intensities of colors; and individual information issued by IC tags or radio or electronic signals from radio wave or electricity tuning circuits or oscillation circuits.

(2) The affinity reaction probe beads according to (1) above, characterized in that the porous carriers are porous glass beads, and the individual identification signals have been written into the surfaces of the beads.

(3) The affinity reaction probe beads according to (1) above, characterized in that the beads each comprise the carrier formed by writing a circuit, which is usable as a radio or electronic recognition system, onto a spherical or tile-shaped silicon crystal to produce the individual identification signal, and then forming a glass layer on the individual identification signal, and have the probe molecules immobilized in the carrier.

(4) The affinity reaction probe beads according to any one of (1) to (3) above, characterized in that the probe molecules are DNA's, RNA's, PNA's and fragments thereof, oligonucleotides having arbitrary base sequences, antigens, antibodies and epitopes, enzymes, proteins and functional site polypeptide chains thereof.

(5) A method for producing affinity reaction probe beads, characterized by immobilizing probe molecules, which have been prepared beforehand, on porous carrier beads with the use of various linkers, the porous carrier beads being provided with one or more individual identification signals among the above-mentioned individual identification signals which are optical signals, or radio or electronic recognition signals.

(6) A method for producing affinity reaction probe beads, characterized by synthesizing oligonucleotides, which are probe molecules, in porous carrier beads marked with one or more individual identification signals among the above-mentioned individual identification signals which are optical signals, or radio or electronic recognition signals.

(7) An affinity reaction probe bead detection system, which places the affinity reaction probe beads according to any one of the above (1) to (4) in a reactor, the affinity reaction probe beads having, immobilized thereto, probe molecules causing different specific binding reactions; bringing the affinity reaction probe beads into contact with an object to be analyzed, thereby causing specific binding; then detecting the presence or absence of binding individually for each of the carriers; and during the detection, detecting the individual identification signals to identify reactions.

The most striking characteristic of the present invention lies in the system which uses many carrier beads having, immobilized therein, different reaction probes each showing a single reaction, reacts these beads with the same sample at the same time, and then detects the reacted beads individually. Under this system, each of the carrier beads having immobilized therein the reaction probes showing single reactions is provided with the individual identification signal, and the probe molecules which have undergone the reactions are confirmed and identified.

Embodiments 1 to 3 according to (1) to (3) above are definitions of the reaction probe beads showing individual single reactions. Embodiment 4 according to (4) above is a definition of the probe molecules used. Embodiments 5 to 6 according to (5) to (6) above are methods for immobilizing the probe molecules. Embodiment 7 according to (7) above shows a mode of actual use for reaction. The contents of the present invention will be explained concretely based on the drawings.

(Structure of and Method for Producing Affinity Reaction Probe Beads)

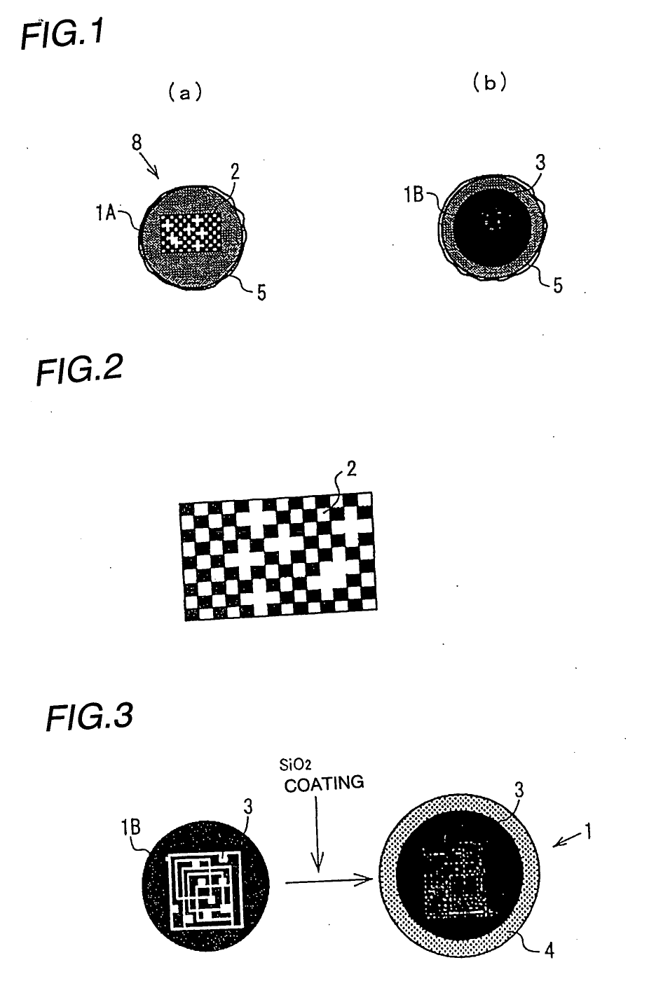

The affinity reaction probe beads of the present invention, as shown in FIGS. 1(a) and 1(b), use a spherical or spheroidal or tile-shaped porous carrier 1 having a diameter of 10 microns to 2 millimeters, preferably 50 microns to 0.5 millimeter. The material for the carrier may be any material which has sufficient physical strength and which is chemically stable. Usually, glass, silica, various synthetic resins, and natural polymers' can be used, and any of these is preferably porous.

The affinity reaction probe beads of the present invention comprise the porous carrier beads, and reactive probe molecules immobilized on the carrier surface or the inner walls of the pores of the porous carrier beads. The porous carrier beads are each provided with one or more individual identification signals among individual identification signals including optical signals such as digital signals using optical graphics, for example, bar codes or dot matrix bar codes 2, and signals utilizing the types and intensities of colors; and radio or electronic signals such as individual information issued by IC tags or those from radio wave or electricity tuning circuits or oscillation circuits. The individual identification signal is provided in a part of each porous carrier bead, and enables the porous carrier beads to be individually identified. The reactive probe molecules recognize particular substances. Two or more of the individual identification signals may be used in combination. The size of the carrier is preferably in the aforementioned range. However, a smaller size than this range may be accepted, if it enables the porous carrier beads to be provided with the above-described individual identification signal, and it enables reactive probe molecules recognizing particular substances to be immobilized therein. During actual use, a large number of reaction probe beads are sequentially flowed through an assay path, and measured. Thus, the smaller size is preferred.

The bead-shaped carrier is preferably porous as a whole, or porous superficially. As shown in FIG. 1(a), for example, the bead-shaped carrier can be prepared by printing an individual recognition mark, as a bar code 2 of a dot matrix, in a part of a particle of porous glass 1A formed by the phase separation method. Alternatively, as shown in FIG. 1(b), the bead-shaped carrier can be prepared by applying a method, in which individual recognition is performed, for example, from a certain resonance frequency, onto single crystal sphere-shaped silicon 1B; or by printing a certain ROM circuit, which can be printed with information under certain conditions, onto the single crystal sphere-shaped silicon 1B. Then, the whole of the so treated carrier is coated with a silica membrane 4 formed by the tetraethoxysilane hydrolysis method which is the so-called sol-gel process (FIG. 3). Of course, the material constituting it may be an organic material such as an ion-exchange resin or cellulose, as long as the material performs the same action.

A probe molecule 5 having reactivity is immobilized, by use of a certain means, onto the surface of the carrier 1 provided beforehand with the particular individual recognition mark, or a surface including the inner surface of the porous structure constituting the surface of the carrier 1. In this case, any material may be immobilized, as long as it is a molecule acting by the mechanism of affinity. Examples of the material are DNA's, RNA's or PNA's (peptide nucleic acids) and their fragments, oligonucleotides having arbitrary base sequences, antigens, antibodies or epitopes, and enzymes, proteins or their functional site polypeptide chains.

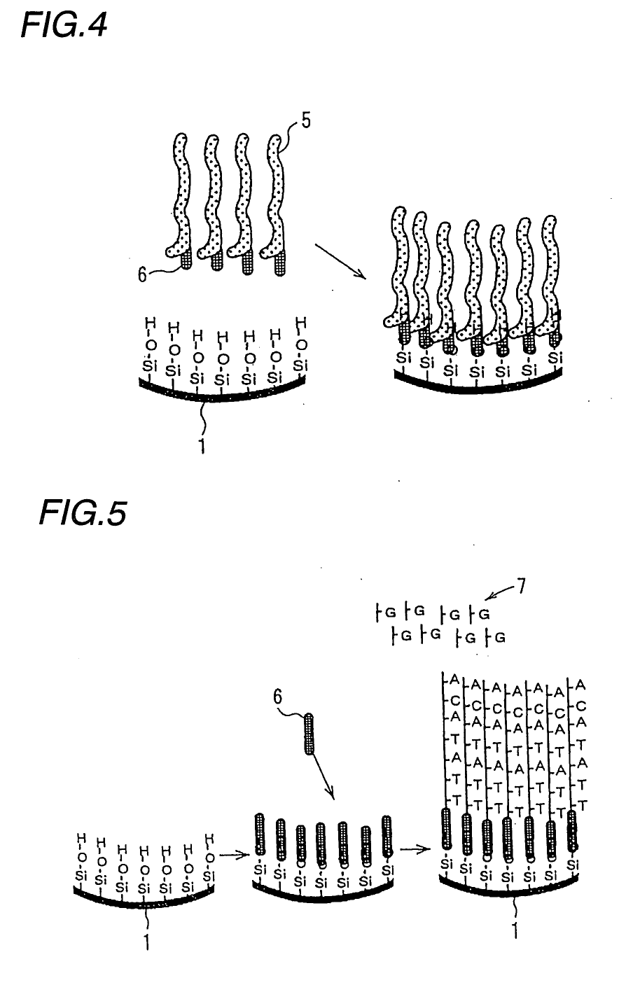

The method of immobilizing the reactive probe molecule 5 onto the carrier 1 preferably comprises, for example, coupling a molecule having a certain binding site, called a linker 6, to the surface of the carrier 1, and fixing the molecule 5, with the linker 6 as an anchor (FIG. 4).

Alternatively, an oligonucleotide may be synthesized in a solid phase on the carrier 1 by use of, for example, the phosphoamidite method to prepare the affinity reaction probe molecule 5 (FIG. 5). The numeral 7 denotes a base block.

These immobilization reactions are excellent in volume production capacity, because they can perform immobilization on the carriers 1 having the same identification number with the use of the same reactor at the same time. Moreover, these probe molecules 5 all have the same characteristics. Thus, their reaction characteristics can be confirmed by a sampling test, and stable product characteristics can be ensured.

As noted above, the affinity reaction probe beads are produced by performing the step of applying the identification signal to the beads, and then carrying out the step of immobilizing the probe molecules on the beads. However, these two steps may be performed in reverse order, unless the production is impeded thereby.

(Reaction, Detection System and Apparatus)

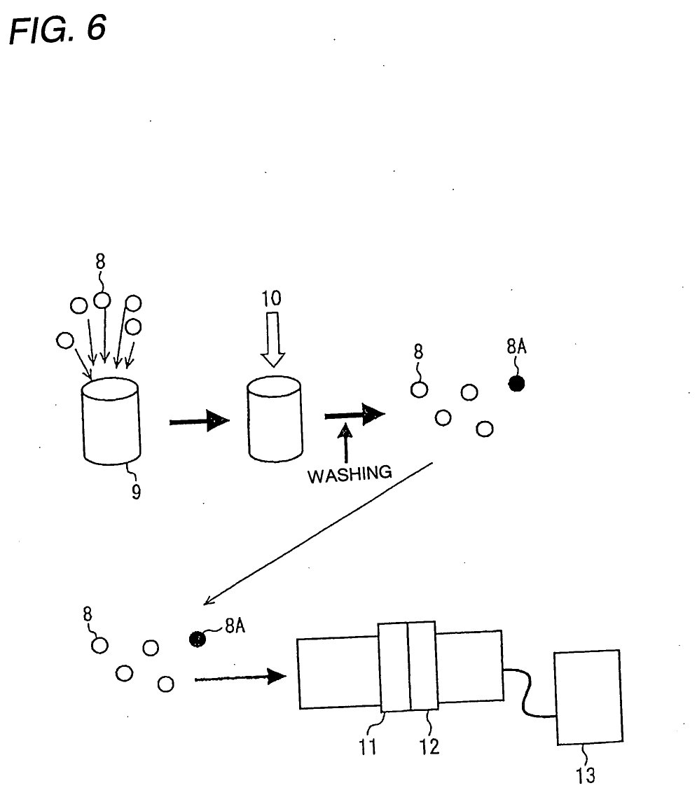

FIG. 6 is a conceptual view of an apparatus for putting the above-described affinity reaction probe beads system into practice.

Affinity reaction probe beads 8 are combined, where necessary, and placed in a reaction tube 9. A sample containing an object to be detected, for example, fluorescence-labeled cDNA, is put into the reaction tube 9. Specific binding of the beads 8 to the analyte is caused by a method such as shaking. After completion of the reaction, the system is washed, and then an operation for detection is performed. The reacted affinity reaction probe beads 8A are subjected to a detector one by one. At this time, the identification signals are read simultaneously. The affinity reaction probe beads 8A, whose reading has been completed, are discarded but, in some cases, preserved, and particular DNA's can be recovered by an extraction operation. The numeral 11 denotes a fluorescence observer, 12 denotes a reader of the identification signal, and 13 denotes a data processor. These instruments put together are designated as the detector. Instead of the fluorescence label, an isotope can be used as a labeling substance.

The procedure, which comprises sequentially feeding the individual affinity reaction probe beads 8A and simultaneously reading the identification signals and also detecting the analytes, produces a series of data. These data are sent to the data processor 13. In this manner, these instruments work as an integral detection system, and function as a gene analysis system similar to a virtual DNA chip.

The present invention will be described further with reference to Examples offered below. However, the present invention is not limited to these Examples. For example, the step of applying the identification signal to the beads, and the step of immobilizing probe molecules on the beads may be performed in reverse order.

### EXAMPLE 1

Porous glass spherical bead carriers of 1 mm in diameter were arrayed, and an amorphous silicon film was vacuum deposited thereon in one direction thereof. A digital identification mark using bar-code optical graphics comprising a certain dot matrix was written into the deposited film by applying photolithography. These carriers were aminated with the use of γ-aminopropyltriethoxysilane. Then, oligonucleotides having particular structures of 20 base pairs were synthesized by the phosphoamidite method using succinic acid as a linker, whereby affinity reaction probe beads were obtained.

These affinity reaction probe beads were placed in a reaction cell of polypropylene, and a sample containing fluorescence-labeled cDNA as an object to be detected was poured into the reaction cell, thereby performing the reaction. After completion of the reaction, the system was washed, and then the reaction probe beads were withdrawn from the cell. The reacted probe beads were analyzed, one by one, by a fluorescence detector and, at the same time, the dot matrices were recognized and identified by a CCD camera.

### EXAMPLE 2

IC tag identification marks composed of certain resonance circuits were formed on single crystal spheres of silicon having a diameter of 0.5 mm. These spheres were each coated with a silica membrane formed by hydrolysis of tetraethoxysilane. The resulting carriers were treated with epoxysilane, which was used as a linker to immobilize particular cDNA's on the carriers.

The thus produced affinity reaction probe beads were placed in a reaction cell of polypropylene, and a sample containing fluorescence-labeled cDNA as an object to be detected was poured into the reaction cell, thereby performing the reaction. After completion of the reaction, the system was washed, and the reaction probe beads were withdrawn from the cell. The reacted probe beads were analyzed, one by one, by a fluorescence detector and, at the same time, individually identified based on resonance frequencies.

### EXAMPLE 3

An individual identification information circuit comprising a certain antenna circuit and an 8-bit writable ROM was formed on each of single crystal spheres of silicon having a diameter of 1 mm. After a protective film was formed on the surface of the spherical silicon, the system was treated with a cuprammonium rayon solution, and also treated with hydrochloric acid to form carriers having a regenerated cellulose layer on the surface. Particular antibodies were adsorbed to and carried on the carriers to prepare affinity reaction probe beads.

The thus produced affinity reaction probe beads were placed in a reaction cell of polypropylene, and a sample containing fluorescence-labeled protein as an object to be detected was poured into the reaction cell, thereby performing the reaction. After completion of the reaction, the system was washed, and the reaction probe beads were withdrawn from the cell. The reacted probe beads were analyzed, one by one, by a fluorescence detector and, at the same time, their individual information was recognized and identified by a read circuit.

### INDUSTRIAL APPLICABILITY

The present invention uses reactive probe substances, such as proteins having arbitrary configurations or oligonucleotides having arbitrary base sequences, and these substances selectively bind to particular molecules, thereby selectively detecting corresponding substances. The present invention also serves as an analytic means for giving information on the existence of many molecules at the same time. Moreover, the present invention can easily provide a detection means capable of high sensitivity analysis without undue burden.

Furthermore, affinity reaction probe beads, which can retain the reactivity of probes in a physically and chemically stable state, are prepared by using porous beads as carriers, and immobilizing probe substances on these carriers. Such advance preparation of unit affinity reaction probe beads by the carriage of various reactive substances on carrier beads makes it possible to provide a reaction detection probe bead analysis system which can be supplied more conveniently using a necessary combination on a necessary occasion, which is low in cost, and which is highly stable.

Hence, it becomes possible to construct a reaction detection probe bead analysis system for DNA, etc. which satisfies the needs of individual persons. This reaction detection probe bead analysis system can contribute to tailor-made medical care.

With the present invention, moreover, the reaction conditions, such as temperature conditions, are easier to control. Thus, unlike conventional DNA chips, the invention provides means for detection in new fields, such as protein detection.

1. Affinity reaction probe beads comprising probe molecules immobilized on carriers which are porous beads provided with one or more individual identification signals.

2. The affinity reaction probe beads according to claim 1, wherein said individual identification signal is selected from an optical signal or a radio or electronic signal.

3. The affinity reaction probe beads according to claim 2, wherein said optical signal is selected from a bar code, a dot matrix and color information.

4. The affinity reaction probe beads according to claim 2, wherein said radio or electronic signal is selected from an IC tag, a tuning circuit and an oscillation circuit.

5. The affinity reaction probe beads according to claim 1, wherein said carriers are porous glass beads.

6. The affinity reaction probe beads according to claim 1, wherein said probe molecules are selected from DNA's, RNA's, PNA's and fragments thereof, oligonucleotides, antigens, antibodies, epitopes, enzymes, proteins and functional site polypeptide chains thereof.

7. The affinity reaction probe beads according to claim 1, wherein principal portions of said probe molecules are bound to inner walls of pores of said porous bead carriers.

8. A method for producing affinity reaction probe beads, characterized by providing porous bead carriers with one or more individual identification signals, and then immobilizing probe molecules, which have been prepared beforehand, on said porous bead carriers with use of a linker.

9. A method for producing affinity reaction probe beads, characterized by providing porous bead carriers with one or more individual identification signals, and then synthesizing oligonucleotides, which are probe molecules, on said carriers.

10. The method for producing affinity reaction probe beads according to claim 8, further comprising binding principal portions of said probe molecules to inner walls of pores of said porous bead carriers.

11. An affinity reaction probe bead detection method, comprising bringing affinity reaction probe beads into contact with an object to be analyzed, thereby causing specific binding; then detecting presence or absence of binding of each of carriers to said object to be analyzed; and during the detection, detecting individual identification signals to identify reactions.

12. An affinity reaction probe bead detection method, comprising:
bringing affinity reaction probe beads into contact with an object to be analyzed, thereby causing specific binding, said affinity reaction probe beads comprising porous bead carriers each provided with one or more different individual recognition signals, and probe molecules immobilized on each of said carriers and causing specific binding reactions different for each of said carriers;
then individually detecting presence or absence of binding of each of said carriers to said object to be analyzed; and
during the detection, detecting individual identification signals to identify reactions.

13. An affinity reaction probe bead detection system, comprising:
a reactor for bringing probe molecules into contact with an object to be analyzed, said probe molecules immobilized on carriers and causing specific binding reactions different for each of said carriers, said carriers being each provided with one or more different individual recognition signals;
a first detector for accepting probe beads after reaction and detecting specific binding in each of said carriers; and
a second detector for detecting individual identification signals.

14. The method for producing affinity reaction probe beads according to claim 9, further comprising binding principal portions of said probe molecules to inner walls of pores of said porous bead carriers.
